(19) United States
(12) Patent Application Publication
(10) Pub. No.: US 2021/0061871 A1
(43) Pub. Date: Mar. 4, 2021

(54) Modified N-810 and Methods Therefor

(71) Applicant: NantBio, Inc., Culver City, CA (US)

(72) Inventors: Kayvan Niazi, Culver City, CA (US); Heather McFarlane, Los Angeles, CA (US)

(21) Appl. No.: 17/006,184

(22) Filed: Aug. 28, 2020

Related U.S. Application Data
(60) Provisional application No. 62/893,662, filed on Aug. 29, 2019.

(51) Int. Cl. C07K 14/54; C07K 14/495; C12N 15/63; C07K 16/28

(52) U.S. Cl. CPC C07K 14/5443; C07K 14/495; C07K 2319/02; C07K 16/2827; C07K 2319/03; C12N 15/63

(57) ABSTRACT

Compositions and methods for multi-specific protein complexes comprising an interleukin-15 (IL-15) domain comprising an N72D mutation (IL-15N72D), a IL-15 receptor alpha sushi-binding domain (IL-15RαSu), an immunoglobulin Fc domain, and a mutated transforming growth factor-beta receptor type 2 (TGFβRII) domain, wherein the mutated TGFβRII domain has a N->Q mutation in positions 47, 71, and 131 respectively. The IL-15RαSu domain, the Fc domain, and the mutated TGFβRII domain are sequentially linked by amide bonds. Preferably, contemplated complexes further include a binding domain that specifically binds to a disease antigen, immune checkpoint molecule, or immune signaling molecule.

This application claims priority to our co-pending US provisional patent application with the Ser. No. 62/893,662, which was filed Aug. 29, 2019, and which is incorporated by reference herein.

### SEQUENCE LISTING

The content of the ASCII text file of the sequence listing named 102719.0021PCT_ST25, which is 134 kb in size was created on Aug. 20, 2020 and electronically submitted via EFS-Web along with the present application is incorporated by reference in its entirety

### FIELD OF THE INVENTION

The field of the invention is multi-specific protein complexes useful in the treatment of a tumor or an infectious disease.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

T×M modifications are promising modifications of the N-803-based IL-15 scaffold. These modifications include the fusion of antibody/ligand sequences which preferentially traffic IL-15 activity to desired sites or tissues in vivo. More recent improvements of the T×M scaffold include the use of the extracellular domain of TGF-β receptors (e.g. “TGF-β traps”) to further functionalize the resulting proteins to compete with native TGF-β receptors at desired sites. However, despite the in vitro demonstration of the validity of this approach, biochemical analysis of the resulting proteins demonstrates a significant amount of glycosylation and non-uniformity in the final product, making industrial commercialization and regulatory approval of such biochemical an unnecessarily risky proposition.

Therefore, there remains a need for compositions and methods to develop new therapeutic molecules that do not have the disadvantages of glycosylation as discussed above.

### SUMMARY OF THE INVENTION

Disclosed herein are various compositions and methods comprising a recombinant protein complex. The recombinant protein complex comprises an interleukin-15 (IL-15) domain comprising an N72D mutation (IL-15N72D), a IL-15 receptor alpha sushi-binding domain (IL-15RαSu), an immunoglobulin Fc domain, and a mutated transforming growth factor-beta receptor type 2 (TGFβRII) domain. The mutated TGFβRII domain is contemplated to have mutated glycosylation sites, preferably the following three mutations: N47Q, N71Q, and N131Q respectively. Furthermore, the IL-15RαSu domain, the Fc domain, and the mutated TGFβRII domain are sequentially linked by amide bonds. The IL-15 domain and/or the IL-15RαSu domain may comprise a binding domain that specifically binds to a disease antigen, immune checkpoint molecule or immune signaling molecule. The IL-15 domain binds to the IL-15RαSu domain to form the recombinant protein complex.

Preferably, the binding domain specifically binds to a programmed death ligand 1 (PD-L1).

In one embodiment, the immunoglobulin Fc domain is linked to the mutated TGFβRII domain via a linker molecule. The TGFβRII domain is contemplated to bind to transforming factor beta (TGFβ). The mutated TGFβRII domain comprises SEQ ID NO: 2

Furthermore, the inventors also contemplate a method of treating a tumor and/or an infectious disease in a subject in need thereof comprising administering to the subject an effective amount of a pharmaceutical composition comprising the recombinant protein complex as disclosed above. The tumor comprises: glioblastoma, prostate cancer, hematological cancer, B-cell neoplasms, multiple myeloma, B-cell lymphoma, B cell non-Hodgkin lymphoma, Hodgkin's lymphoma, chronic lymphocytic leukemia, acute myeloid leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, prostate cancer, pancreatic cancer, colorectal cancer, ovarian cancer, non-small cell lung carcinoma, or squamous cell head and neck carcinoma. Optionally, a second therapeutic agent, for example a chemotherapeutic agent, may be administered to the subject.

In one embodiment, disclosed herein is a method of inducing antibody-dependent cell-mediated cytotoxicity (ADCC) in a subject in need thereof, comprising administering to a subject in need thereof, an effective amount of the recombinant protein complex disclosed herein.

In another aspect, disclosed herein is an expression vector encoding the recombinant protein complex. The expression vector may be a viral vector, a bacterial vector, or a yeast vector. Preferably, the viral vector is an adenoviral vector. In especially preferred embodiments, the adenoviral vector has E1 and E2b genes deleted.

In one embodiment, disclosed herein is a use of a viral expression vector for the treatment a tumor and/or an infectious disease in a subject in need, the viral expression vector comprising a first segment encoding an interleukin-15 (IL-15) domain comprising an N72D mutation (IL-15N72D); and a second segment encoding a polypeptide comprising a binding domain that specifically binds to a disease antigen, immune checkpoint molecule or immune signaling molecule, wherein the binding domain is linked to a IL-15 receptor alpha sushi-binding domain (IL-15RαSu) that is linked to an immunoglobulin Fc domain which is linked to a mutated transforming growth factor-beta receptor type 2 (TGFβRII) domain, wherein the mutated TGFβRII domain has a N->Q mutation in positions 47, 71, and 131 respectively. In preferred embodiments, the vector is a viral vector, for example a viral vector adenoviral vector. The adenovirus may have E1 and E2b genes deleted. In other embodiments, the vector may also be a yeast expression vector, or a bacterial expression vector. In one embodiment, the immunoglobulin Fc domain is linked to a transforming growth factor-beta receptor type 2 (TGFβRII) domain via a linker molecule. In one embodiment, the binding domain specifically binds to one or more molecules comprising: programmed death ligand 1 (PD-L1). In some embodiments, the binding domain specifically binds to one or more molecules comprising: programmed death ligand 1 (PD-L1), programmed death 1 (PD-1), cytotoxic T-lymphocyte associated protein 4 (CTLA-4), cluster of differentiation 33 (CD33), cluster of differentiation 47 (CD47), glucocorticoid-induced tumor necrosis factor receptor (TNFR) family related gene (GITR), lymphocyte function-associated antigen 1 (LFA-1), tissue factor (TF), delta-like protein 4 (DLL4), single strand DNA or T-cell immunoglobulin and mucin-domain containing-3 (Tim-3). In some embodiments, the TGFβRII domain binds to transforming factor beta (TGFβ) and/or the mutated TGFβRII domain comprises SEQ ID NO: 2

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates that there are 3 possible N-glycosylation sites in TGFβRII, and 6 extra N-glycosylation sites in total in huPD-L1/T×M/TGFβRII.

FIG. 2 illustrates that heterogeneity huPD-L1/T×M/TGFβRII likely represents different glycosylation patterns and various occupancies.

FIG. 3 depicts that both the wild type Rsbc6/T×M/TGFβRII(WT) protein complex, and the N47Q variant thereof, wherein the N47Q mutation is on TGFβRII, have multiple peaks due to glycosylation on TGFβRII.

FIG. 4 depicts various protein constructs used in the instant study and disclosure.

FIG. 5 illustrates that aglycosylated TGFβRII designed with N→Q mutations in positions 47, 71, and 131 results in the same glycosylation pattern as N-809A and yields a homogeneous product.

### DETAILED DESCRIPTION

The inventors have now discovered that while multi-specific IL-15-based protein complexes, such as N-803, T×M, modified N-803, or modified T×M (as disclosed in US Publication No.: US20200002425A1, which is incorporated by reference herein) enhance the activity of immune cells and promote their activity against disease cells, thereby resulting in reduction or prevention of disease, nevertheless face disadvantages. Throughout this disclosure, by “T×M” is meant a complex comprising an IL-15N72D:IL-15RαSu/Fc scaffold linked to a binding domain. An exemplary T×M is an IL-15N72D:IL-15RαSu/Fc complex comprising a fusion to a binding domain that specifically recognizes PD-L1 (PD-L1 T×M).

The US Publication No.: US20200002425A1 disclose a T×M scaffold that includes the extracellular domain of TGF-β receptors (e.g. “TGF-β traps”) to further functionalize the resulting proteins to compete with native TGF-β receptors at desired sites. However, despite the in vitro demonstration of the validity of this approach, biochemical analysis of the resulting proteins demonstrated a significant amount of glycosylation and non-uniformity in the final product, making industrial commercialization and regulatory approval of such biochemical problematic.

As disclosed herein, the inventors overcame these complications by genetically modifying the TGF-β trap portion of the proteins to produce aglycosylated versions of these proteins which retained biological activity in vitro and in vivo. In one embodiment, the engineering of aglycosylated cytokine receptor traps can be applied to other TGF-β systems or other cytokine receptor fusions (e.g. TNF-α competing agents like Etanercept, etc).

One aspect of the present disclosure provides a recombinant protein complex comprising: an interleukin-15 (IL-15) domain comprising an N72D mutation (IL-15N72D), a IL-15 receptor alpha sushi-binding domain (IL-15RαSu), an immunoglobulin Fc domain, and a mutated transforming growth factor-beta receptor type 2 (TGFβRII) domain, wherein the mutated TGFβRII domain has a N->Q mutation in positions 47, 71, and 131 respectively. The IL-15RαSu domain, the Fc domain, and the mutated TGFβRII domain are contemplated to be sequentially linked by amide bonds to form a single polypeptide chain. Preferably, the IL-15RαSu domain is further linked to an anti PD-L1 scFv. It is further contemplated that the IL-15 domain and/or the IL-15RαSu domain comprises a binding domain that specifically binds to a disease antigen, immune checkpoint molecule or immune signaling molecule, and wherein the IL-15 domain binds to the IL-15RαSu domain to form the recombinant protein complex.

The protein complexes disclosed herein show increased binding to disease and target antigens. Such protein complexes have utility in methods for treating a neoplasia, infectious disease, or autoimmune disease in a subject. Thus, provided herein are compositions featuring anti-PD-L1/TGFβRII/T×M and methods of using such compositions to enhance an immune response against a tumor (e.g., solid and hematologic tumors).

In certain embodiments, the immunoglobulin Fc domain is linked to a transforming growth factor-beta receptor type 2 (TGFβRII) domain via a linker molecule. The linker sequence should be flexible and allow effective positioning of the immunoglobulin Fc domain with respect to the TGFβRII to allow functional activity of both domains. Furthermore, the recombinant protein complexes may also have a linker between the IL-15 or IL-15Rα domains and the biologically active polypeptide. As before, the linker sequence should allow effective positioning of the biologically active polypeptide with respect to the IL-15 or IL-15Rα domains to allow functional activity of both domains. Preferably, the linker sequence comprises from about 7 to 20 amino acids, more preferably from about 10 to 20 amino acids. The linker sequence is preferably flexible so as not hold the two biologically active molecule that is being linked in a single undesired conformation.

In various embodiments, the binding domain of the recombinant protein complex specifically binds to one or more molecules comprising: programmed death ligand 1 (PD-L1), programmed death 1 (PD-1), cytotoxic T-lymphocyte associated protein 4 (CTLA-4), cluster of differentiation 33 (CD33), cluster of differentiation 47 (CD47), glucocorticoid-induced tumor necrosis factor receptor (TNFR) family related gene (GITR), lymphocyte function-associated antigen 1 (LFA-1), tissue factor (TF), delta-like protein 4 (DLL4), single strand DNA or T-cell immunoglobulin and mucin-domain containing-3 (Tim-3). In these embodiments, the binding domain comprises anti-PD-L1, anti-PD-1, anti-CTLA-4, anti-CD33, anti-CD4, anti-TNFR family related gene (GITR), anti-LFA-1, anti-TF, and anti-DLL4, anti-Tim-3 respectively.

In an especially preferred embodiment, the binding domain comprises an anti-PD-L1 antibody. In this particular embodiment, the binding domain of the recombinant protein complex specifically binds to one or more molecules of programmed death ligand 1 (PD-L1).

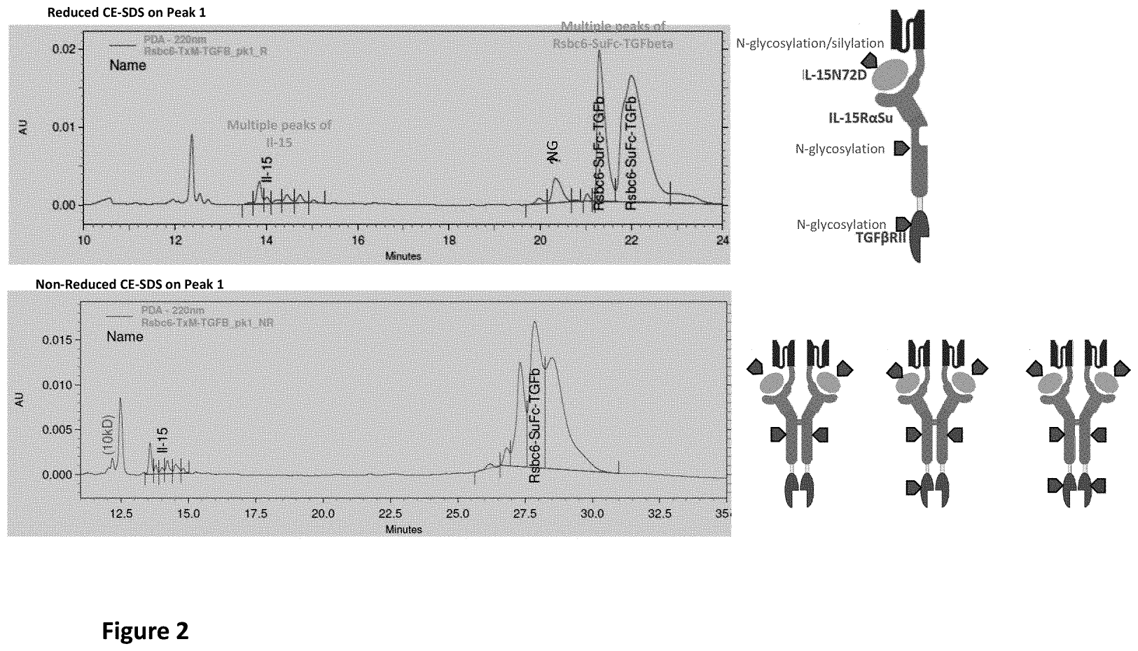

The TGFβRII domain of the recombinant protein complex is contemplated to be mutated to prevent glycosylation. As shown in FIG. 2, the use of wild type TGFβRII domain in anti-huPD-L1/T×M/TGFβRII results in heterogeneity, likely due to different glycosylation patterns and various occupancies. As shown in both the native and the reduced CS-SDS gels multiple peaks are seen for IL-15 and Rsbc6-SuFc-TGFβ. These multiple peaks show the non-uniformity of the final product, which is most likely due to significant amount of glycosylation. This non-uniformity makes industrial commercialization and regulatory approval of such biochemical an unnecessarily risky proposition.

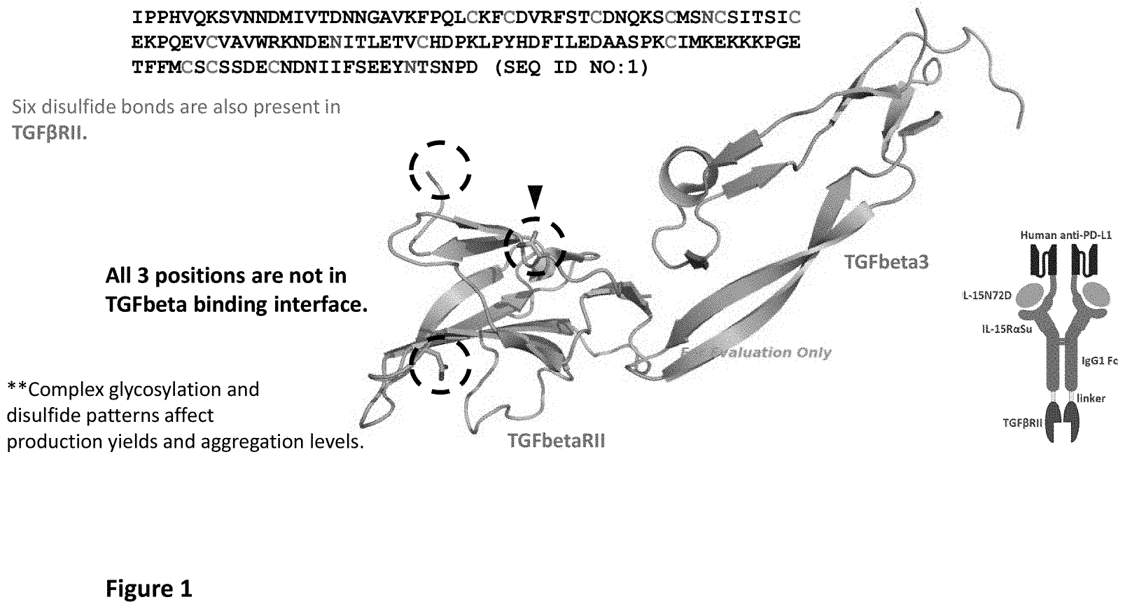

The inventors solved this problem by making mutated TGFβRII constructs. The wild and mutated polypeptide sequences of TGFβRII domain are shown in SEQ ID NO: 1 and SEQ ID NO: 2 respectively. As illustrated in FIG. 1, there are three possible N-glycosylation sites in TGFβRII, and six extra N-glycosylation sites in total in anti-huPD-L1/T×M/TGFβRII. Furthermore, there are six disulfide bonds also present in TGFβRII. It is known that complex glycosylation and disulfide patterns affect production yields and aggregation levels. The inventors sought to make mutations in the TGFβRII polypeptide that did not inhibit or reduce the biological activity of the polypeptide, but ensured that the glycosylation sites were mutated so as to lead to a uniform final product.

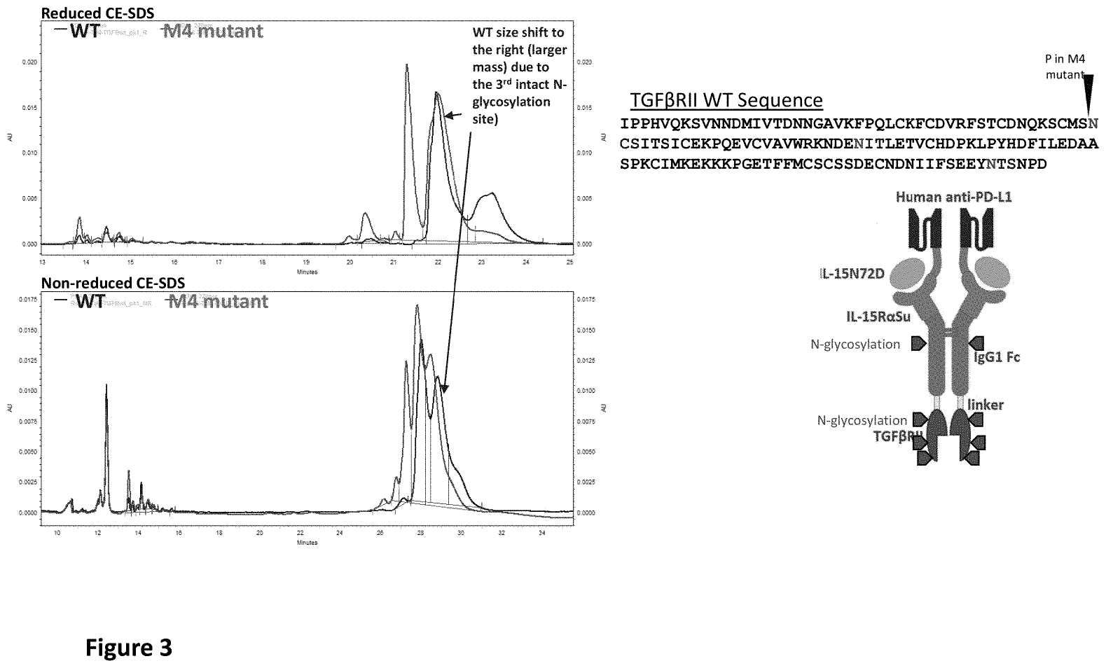

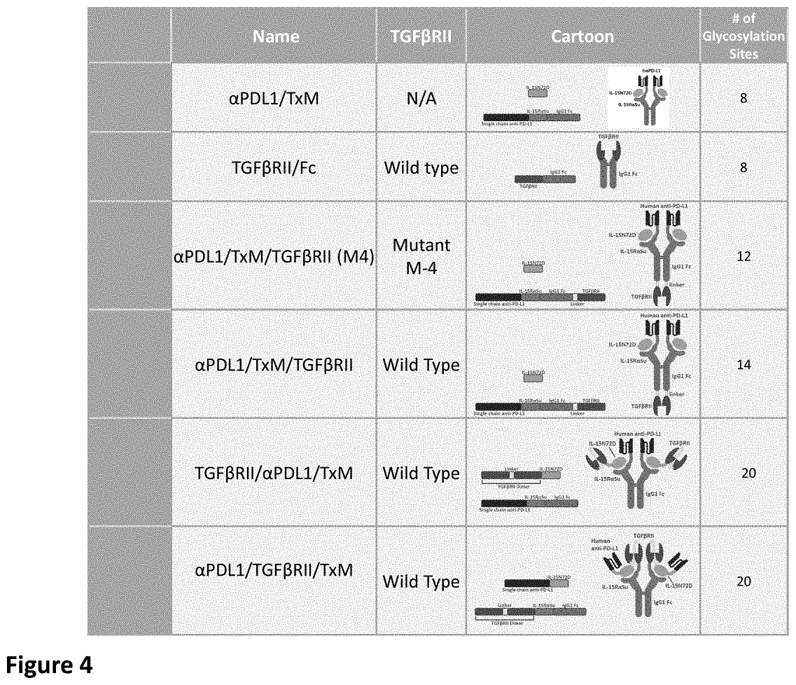

With the N47P mutation, as illustrated in FIG. 3, both the wild type (Rsbc6/T×M/TGFβRII) and the N47P mutated construct had multiple peaks due to glycosylation on TGFβRII. Furthermore, the weight size shifted to the right (larger mass) due to the 3^{rd} intact N-glycosylation site. As this mutation was unsuccessful, the inventors designed several more mutated constructs, some of which are shown in FIG. 4.

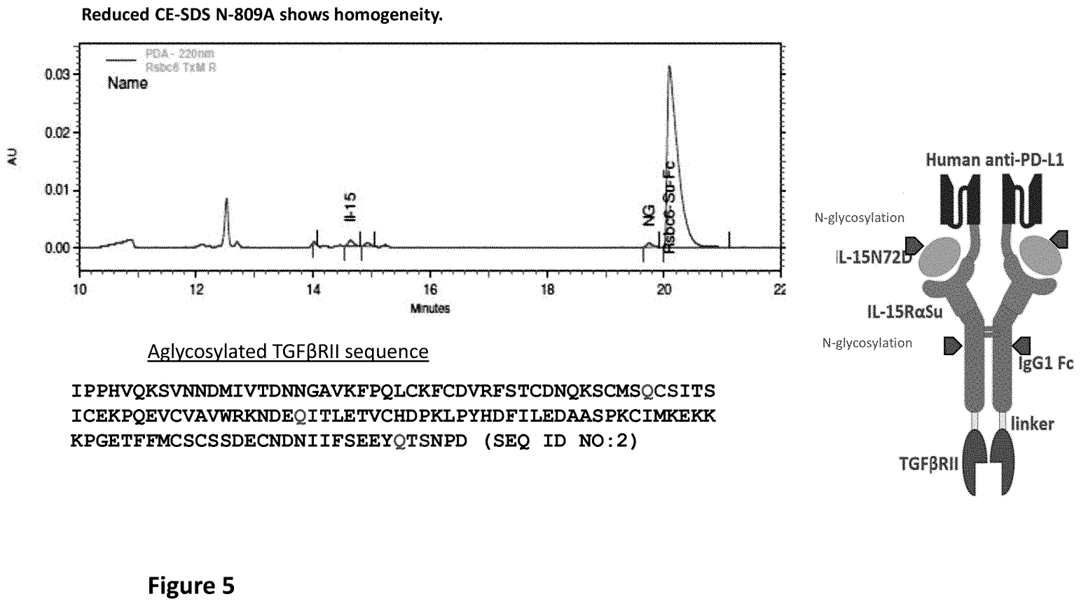

With the N47Q, N71Q, and N131Q mutations on the TGFβRII polypeptide, the inventors found the Rsbc6/T×M/TGFβRII mutated construct led to a single homogenous product, as shown in FIG. 5.

As described herein, the use of proteins with the capability of targeting diseased cells for host immune recognition and response is an effective strategy for treating cancer, infectious diseases, and autoimmune diseases. As described in U.S. Pat. No. 8,507,222 (incorporated herein by reference), a protein scaffold comprising IL-15 and IL-15 receptor a domains has been used to generate multi-specific proteins capable of recognizing antigens on disease cells and receptors on immune cells.

In some cases, these complexes also comprise binding domains that recognize antigens, such as PD-L1, ssDNA, CD20, HER2, EGFR, CD19, CD38, CD52, GD2, CD33, Notch1, intercellular adhesion molecule 1 (ICAM-1), tissue factor, HIV envelope or other tumor antigens, expressed on disease cells.

In some cases, the multi-specific recombinant protein complexes further comprise an IgG Fc domain for protein dimerization and recognition of CD16 receptors on immune cells. Such a domain mediates stimulation of antibody-dependent cellular cytotoxicity (ADCC), antibody-dependent cellular phagocytosis (ADCP) and complement-dependent cytotoxicity (CDC) against target cells. In some examples, it is useful to employ Fc domains with enhanced or decreased CD16 binding activity. In one aspect, the Fc domain contains amino acid substitutions L234A and L235A (LALA) (number based on Fc consensus sequence) that reduce ADCC activity but retain the ability to form disulfide-bound dimers.

Accordingly, in certain embodiments, the recombinant protein complex comprises at least two protein complexes, a first protein complex comprises an interleukin-15 (IL-15 or IL15 mutant such as N72D) polypeptide domain and a second protein comprises a soluble IL-15 receptor alpha sushi-binding domain (IL-15RαSu) fused to an immunoglobulin Fc domain, wherein the immunoglobulin Fc domain is fused or linked to a transforming growth factor-beta receptor type 2 (TGFβRII) domain; the first and/or second soluble protein further comprises a binding domain that specifically binds to a disease antigen, immune checkpoint molecule or immune signaling molecule, and the IL-15 domain of the first protein binds to the IL-15RαSu domain of the second soluble protein to form a fusion protein complex. In certain aspects, the immunoglobulin Fc domain is linked to a transforming growth factor-beta receptor type 2 (TGFβRII) domain via a linker molecule.

In certain embodiments, one of the first or second soluble protein further comprises a second binding domain (preferably distinct from the first binding domain) that specifically binds to a disease antigen, immune checkpoint molecule, or immune signaling molecule.

Also disclosed herein is a method of treating a tumor and/or an infectious disease in a subject in need thereof comprising administering to the subject an effective amount of a pharmaceutical composition comprising the recombinant protein complex. The tumor may comprise glioblastoma, prostate cancer, hematological cancer, B-cell neoplasms, multiple myeloma, B-cell lymphoma, B cell non-Hodgkin lymphoma, Hodgkin's lymphoma, chronic lymphocytic leukemia, acute myeloid leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, prostate cancer, pancreatic cancer, colorectal cancer, ovarian cancer, non-small cell lung carcinoma, or squamous cell head and neck carcinoma.

The pharmaceutical composition comprising the recombinant protein complex is administered in an effective amount. For example, an effective amount of the pharmaceutical composition is between about 1 μg/kg and 100 μg/kg, e.g., 1, 5, 10, 15, 20, 25, 30, 35, 40, 45, 50, 55, 60, 65, 70, 75, 80, 85, 90, 95, or 100 μg/kg. Alternatively, the mutated T×M complex is administered as a fixed dose or based on body surface area (i.e., per m²).

The pharmaceutical composition comprising the recombinant protein complex is administered at least one time per month, e.g., twice per month, once per week, twice per week, once per day, twice per day, every 8 hours, every 4 hours, every 2 hours, or every hour. Suitable modes of administration for the pharmaceutical composition include systemic administration, intravenous administration, local administration, subcutaneous administration, intramuscular administration, intratumoral administration, inhalation, and intraperitoneal administration.

The methods of treatment contemplated herein may further, optionally, comprise administering to the subject one or more chemotherapeutic agents. Some non-limiting examples of chemotherapeutic agents contemplated herein are vindesine, vincristine, vinblastin, methotrexate, adriamycin, bleomycin, or cisplatin.

In another aspect, contemplated herein is an expression vector encoding the recombinant protein complex disclosed herein. The expression vector may be a viral expression vector or a yeast expression vector. In this context it should be recognized that the expression vector may be used for in vitro expression and production of the protein complexes following conventional recombinant expression protocols, or the vector may be used for in vivo production where the individual to be treated is provided with the vector (e.g., viral vector in a recombinant virus) that leads to in vivo expression of the protein complexes.

The vectors will typically comprise a recombinant nucleic acid that encodes a protein complex that comprises an interleukin-15 (IL-15) domain comprising an N72D mutation (IL-15N72D), and a IL-15 receptor alpha sushi-binding domain (IL-15RαSu) linked to an immunoglobulin Fc domain which is linked to a mutated transforming growth factor-beta receptor type 2 (TGFβRII) domain, wherein the mutated TGFβRII domain has the following three mutations N47Q, N71Q, and N131Q. The IL-15 domain and/or the IL-15RαSu domain comprises a binding domain that specifically binds to a disease antigen, immune checkpoint molecule or immune signaling molecule. The IL-15 domain binds to the IL-15RαSu domain to form a recombinant protein complex. In especially preferred embodiments, the binding domain is anti-PD-L1, and the anti-PD-L1 is covalently linked to the IL-15RαSu domain.

With respect to recombinant viruses it is contemplated that all known manners of making recombinant viruses are deemed suitable for use herein, however, especially preferred viruses are those already established in therapy, including adenoviruses, adeno-associated viruses, alphaviruses, herpes viruses, lentiviruses, etc. Among other appropriate choices, adenoviruses are particularly preferred.

Moreover, it is further generally preferred that the virus is a replication deficient and non-immunogenic virus. For example, suitable viruses include genetically modified alphaviruses, adenoviruses, adeno-associated viruses, herpes viruses, lentiviruses, etc. However, adenoviruses are particularly preferred. For example, genetically modified replication defective adenoviruses are preferred that are suitable not only for multiple vaccinations but also vaccinations in individuals with preexisting immunity to the adenovirus (see e.g., WO 2009/006479 and WO 2014/031178, which are incorporated by reference in its entirety). In some embodiments, the replication defective adenovirus vector comprises a replication defective adenovirus 5 vector. In some embodiments, the replication defective adenovirus vector comprises a deletion in the E2b region. In some embodiments, the replication defective adenovirus vector further comprises a deletion in the E1 region. In that regard, it should be noted that deletion of the E2b gene and other late proteins in the genetically modified replication defective adenovirus to reduce immunogenicity. Moreover, due to these specific deletions, such genetically modified viruses were replication deficient and allowed for relatively large recombinant cargo.

For example, WO 2014/031178 describes the use of such genetically modified viruses to express CEA (colorectal embryonic antigen) to provide an immune reaction against colon cancer. Moreover, relatively high titers of recombinant viruses can be achieved using genetically modified human 293 cells as has been reported (e.g., J Virol. 1998 February; 72(2): 926-933).

E1-deleted adenovirus vectors Ad5 [E1-] are constructed such that a trans gene replaces only the E1 region of genes. Typically, about 90% of the wild-type Ad5 genome is retained in the vector. Ad5 [E1−] vectors have a decreased ability to replicate and cannot produce infectious virus after infection of cells not expressing the Ad5 E1 genes. The recombinant Ad5 [E1−] vectors are propagated in human cells allowing for Ad5 [E1−] vector replication and packaging. Ad5 [E1−] vectors have a number of positive attributes; one of the most important is their relative ease for scale up and cGMP production. Currently, well over 220 human clinical trials utilize Ad5 [E1−] vectors, with more than two thousand subjects given the virus sc, im, or iv. Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1-] vectors have a carrying capacity that approaches 7 kb.

One obstacle to the use of first generation (E1-deleted) Ad5-based vectors is the high frequency of pre-existing anti-adeno virus type 5 neutralizing antibodies. Attempts to overcome this immunity is described in WO 2014/031178, which is incorporated by reference herein. Specifically, a novel recombinant Ad5 platform has been described with deletions in the early 1 (E1) gene region and additional deletions in the early 2b (E2b) gene region (Ad5 [E1−, E2b−]). Deletion of the E2b region (that encodes DNA polymerase and the preterminal protein) results in decreased viral DNA replication and late phase viral protein expression. E2b deleted adenovirus vectors provide an improved Ad-based vector that is safer, more effective, and more versatile than First Generation adenovirus vectors.

In a further embodiment, the adenovirus vectors contemplated for use in the present disclosure include adenovirus vectors that have a deletion in the E2b region of the Ad genome and, optionally, deletions in the E1, E3 and, also optionally, partial or complete removal of the E4 regions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase and/or the preterminal protein functions deleted.

Therefore, and regardless of the type of recombinant virus it is contemplated that the virus may be used to infect patient (or non-patient) cells ex vivo or in vivo. For example, the virus may be injected subcutaneously or intravenously, or may be administered intranasaly or via inhalation to so infect the patient's cells, and especially antigen presenting cells. Alternatively, immune competent cells (e.g., NK cells, T cells, macrophages, dendritic cells, etc.) of the patient (or from an allogeneic source) may be infected in vitro and then transfused to the patient. Alternatively, immune therapy need not rely on a virus but may be effected with nucleic acid transfection or vaccination using RNA or DNA, or other recombinant vector that leads to the expression of the neoepitopes (e.g., as single peptides, tandem mini-gene, etc.) in desired cells, and especially immune competent cells.

As noted above, the desired nucleic acid sequences (for expression from virus infected cells) are under the control of appropriate regulatory elements well known in the art. For example, suitable promoter elements include constitutive strong promoters (e.g., SV40, CMV, UBC, EF1A, PGK, CAGG promoter), but inducible promoters are also deemed suitable for use herein, particularly where induction conditions are typical for a tumor microenvironment. For example, inducible promoters include those sensitive to hypoxia and promoters that are sensitive to TGF-β or IL-8 (e.g., via TRAF, JNK, Erk, or other responsive elements promoter). In other examples, suitable inducible promoters include the tetracycline-inducible promoter, the myxovirus resistance 1 (M×1) promoter, etc.

The replication defective adenovirus comprising an E1 gene region deletion, an E2b gene region deletion, and a nucleic acid encoding the recombinant protein complex as described herein may be administered to a patient in need for inducing immunity against a tumor. Routes and frequency of administration of the therapeutic compositions described herein, as well as dosage, may vary from individual to individual, and the severity of the disease, and may be readily established using standard techniques. In some embodiments, the administration comprises delivering 4.8-5.2×10¹¹ replication defective adenovirus particles, or 4.9-5.1×10¹¹ replication defective adenovirus particles, or 4.95-5.05×10¹¹ replication defective adenovirus particles, or 4.99-5.01×10¹¹ replication defective adenovirus particles.

The administration of the virus particles can be through a variety of suitable paths for delivery. One preferred route contemplated herein is by injection, such as intratumoral injection, intramuscular injection, intravenous injection or subcutaneous injection. In some embodiments, a subcutaneous delivery may be preferred.

With respect to yeast expression and vaccination systems, it is contemplated that all known yeast strains are deemed suitable for use herein. However, it is preferred that the yeast is a recombinant Saccharomyces strain that is genetically modified with a nucleic acid construct encoding a protein complex as presented herein, to thereby initiate an immune response against the tumor. In one aspect of any of the embodiments of the disclosure described above or elsewhere herein, the yeast vehicle is a whole yeast. The whole yeast, in one aspect is killed. In one aspect, the whole yeast is heat inactivated. In one preferred embodiment, the yeast is a whole, heat-inactivated yeast from Saccharomyces cerevisiae.

The use of a yeast based therapeutic compositions are disclosed in the art. For example, WO 2012/109404 discloses yeast compositions for treatment of chronic hepatitis b infections.

It is noted that any yeast strain can be used to produce a yeast vehicle of the present disclosure. Yeasts are unicellular microorganisms that belong to one of three classes: Ascomycetes, Basidiomycetes and Fungi Imperfecti. One consideration for the selection of a type of yeast for use as an immune modulator is the pathogenicity of the yeast. In preferred embodiments, the yeast is a non-pathogenic strain such as Saccharomyces cerevisiae as non-pathogenic yeast strains minimize any adverse effects to the individual to whom the yeast vehicle is administered. However, pathogenic yeast may also be used if the pathogenicity of the yeast can be negated using pharmaceutical intervention.

For example, suitable genera of yeast strains include Saccharomyces, Candida, Cryptococcus, Hansenula, Kluyveromyces, Pichia, Rhodotorula, Schizosaccharomyces and Yarrowia. In one aspect, yeast genera are selected from Saccharomyces, Candida, Hansenula, Pichia or Schizosaccharomyces, and in a preferred aspect, Saccharomyces is used. Species of yeast strains that may be used include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Rhodotorula rubra, Schizosaccharomyces pombe, and Yarrowia lipolytica.

Transfection of a nucleic acid molecule into a yeast cell according to the present disclosure can be accomplished by any method by which a nucleic acid molecule administered into the cell and includes diffusion, active transport, bath sonication, electroporation, microinjection, lipofection, adsorption, and protoplast fusion. Transfected nucleic acid molecules can be integrated into a yeast chromosome or maintained on extrachromosomal vectors using techniques known to those skilled in the art. As discussed above, yeast cytoplast, yeast ghost, and yeast membrane particles or cell wall preparations can also be produced recombinantly by transfecting intact yeast microorganisms or yeast spheroplasts with desired nucleic acid molecules, producing the antigen therein, and then further manipulating the microorganisms or spheroplasts using techniques known to those skilled in the art to produce cytoplast, ghost or subcellular yeast membrane extract or fractions thereof containing desired antigens or other proteins. Further exemplary yeast expression systems, methods, and conditions suitable for use herein are described in US20100196411A1, US2017/0246276, or US 2017/0224794, and US 2012/0107347.

So produced recombinant viruses and yeasts may then be individually or in combination used as a therapeutic vaccine in a pharmaceutical composition, typically formulated as a sterile injectable composition with a virus of between 10⁴-10¹³ virus or yeast particles per dosage unit, or more preferably between 10⁹-10¹² virus or yeast particles per dosage unit. Alternatively, virus or yeast may be employed to infect patient cells ex vivo and the so infected cells are then transfused to the patient. However, alternative formulations are also deemed suitable for use herein, and all known routes and modes of administration are contemplated herein.

### Sequences

Various exemplary sequences of the modified N-810 recombinant protein complex are shown below.

N-810A: In one embodiment, the recombinant protein complex disclosed herein comprises human αPDL1/T×M/TGFβRII (M4 variant). In this embodiment, the polypeptide sequences of SEQ ID NO: 3 and SEQ ID NO: 4 are stabilized by hydrophobic or hydrophilic interactions to form the N-810A recombinant protein complex. SEQ ID NO: 3 comprises, in a sequential manner, Leader Peptide, human αPDL1 scFv, IL15Rα-Fc, (G4S)₄ Linker, and human TGFβRII. SEQ ID NO: 4 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:3-4.

N-810B: In one embodiment, the recombinant protein complex disclosed herein comprises human (TGFβRII dimer/human αPDL1/T×M). In this embodiment, the polypeptide sequences of SEQ ID NO: 5 and SEQ ID NO: 6 are stabilized by hydrophobic or hydrophilic interactions to form the N-810B recombinant protein complex. SEQ ID NO: 5 comprises, in a sequential manner, Leader Peptide, human αPDL1 scFv, and IL15Rα-Fc. SEQ ID NO: 6 comprises, in a sequential manner, Leader Peptide, human TGFβRII dimer, and IL15 (N72D). Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:5-6.

N-810 C: In one embodiment, the recombinant protein complex disclosed herein comprises human αPDL1/TGFβRII dimer/T×M. In this embodiment, the polypeptide sequences of SEQ ID NO: 7 and SEQ ID NO: 8 are stabilized by hydrophobic or hydrophilic interactions to form the N-810C recombinant protein complex. SEQ ID NO: 7 comprises, in a sequential manner, Leader Peptide, human TGFβRII dimer, and IL15Rα-Fc. SEQ ID NO: 8 comprises, in a sequential manner, Leader Peptide, human αPDL1 scFv, and IL15 (N72D). Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:7-8.

N-810D: In one embodiment, the recombinant protein complex disclosed herein comprises N-810 (h2*αPDL1/T×M/TGFβRII-WT). In this embodiment, the polypeptide sequences of SEQ ID NO: 9 and SEQ ID NO: 10 are stabilized by hydrophobic or hydrophilic interactions to form the recombinant protein complex. SEQ ID NO: 9 comprises, in a sequential manner, Leader Peptide, human αPDL1 scFv, IL15Rα-Fc, (G4S)4 Linker, and human TGFβRII. SEQ ID NO: 10 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:9-10.

N-810E: In one embodiment, the recombinant protein complex disclosed herein comprises human αPDL1/TGFβRII/T×M. In this embodiment, the polypeptide sequences of SEQ ID NO: 11 and SEQ ID NO: 12 are stabilized by hydrophobic or hydrophilic interactions to form the recombinant protein complex. SEQ ID NO: 11 comprises, in a sequential manner, Leader Peptide, human TGFβRII, and IL15Rα-Fc. SEQ ID NO: 12 comprises, in a sequential manner, Leader Peptide, human αPDL1 scFv, and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:11-12.

N-810 A delta C: In one embodiment, the recombinant protein complex disclosed herein comprises N-810 A delta C. In this embodiment, the polypeptide sequences of SEQ ID NO: 13 and SEQ ID NO: 14 are stabilized by hydrophobic or hydrophilic interactions to form the recombinant protein complex. SEQ ID NO: 13 comprises, in a sequential manner, Leader Peptide, human αPDL1 scFv, IL15Rα-Fc-C312S, (G45)4 Linker, and human TGFβRII. SEQ ID NO: 4 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:13-14.

N-810 A delta C (TGFβRII-aglycosylated): In one embodiment, the recombinant protein complex disclosed herein comprises N-810 A delta C (TGFβRII-aglycosylated). In this embodiment, the polypeptide sequences of SEQ ID NO: 15 and SEQ ID NO: 16 are stabilized by hydrophobic or hydrophilic interactions to form the recombinant protein complex. SEQ ID NO: 15 comprises, in a sequential manner, Leader Peptide, human αPDL1 scFv, IL15Rα-Fc-C312S, and human TGFβRII-N607Q, N631Q, N691Q. SEQ ID NO: 16 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:15-16.

N-810 D: In one embodiment, the recombinant protein complex disclosed herein comprises N-810 D. In this embodiment, the polypeptide sequences of SEQ ID NO: 17 and SEQ ID NO: 18 are stabilized by hydrophobic or hydrophilic interactions to form the recombinant protein complex. SEQ ID NO: 17 comprises, in a sequential manner, Leader Peptide, IL15Rα-Fc, (G45)4 Linker, and human TGFβRII. SEQ ID NO: 18 comprises, in a sequential manner, Leader Peptide, human αPDL1 scFv, and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:17-18.

N-810 A (h2*αPDL1/T×M/TGRβRII-aglycosylated): In one embodiment, the recombinant protein complex disclosed herein comprises N-810 A (h2*αPDL1/T×M/TGRβRII-aglycosylated). In this embodiment, the polypeptide sequences of SEQ ID NO: 19 and SEQ ID NO: 20 are stabilized by hydrophobic or hydrophilic interactions to form the recombinant protein complex. SEQ ID NO: 19 comprises, in a sequential manner, Leader Peptide, human αPDL1 scFv, IL15Rα-Fc, (G45)4 Linker, and human TGFβRII-N607Q, N631Q, N691Q. SEQ ID NO: 20 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:19-20.

N 810 A Delta Hinge: In one embodiment, the recombinant protein complex disclosed herein comprises N 810 A Delta Hinge. In this embodiment, the polypeptide sequences of SEQ ID NO: 21 and SEQ ID NO:22 are stabilized by hydrophobic or hydrophilic interactions to form the recombinant protein complex. SEQ ID NO:21 comprises, in a sequential manner, Leader Peptide, human αPDL1 scFv, IL15Rα-Fc, (G45)4 Linker, and human TGFβRII. SEQ ID NO:22 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:21-22.

N 810 A (IL15-M38): In one embodiment, the recombinant protein complex disclosed herein comprises N 810 A (IL15-M38). In this embodiment, the polypeptide sequences of SEQ ID NO: 23 and SEQ ID NO: 24 are stabilized by hydrophobic or hydrophilic interactions to form the recombinant protein complex. SEQ ID NO: 23 comprises, in a sequential manner, Leader Peptide, human αPDL1 scFv, IL15Rα-Fc, (G45)4 Linker, and human TGFβRII. SEQ ID NO: 24 comprises, in a sequential manner, Leader Peptide and IL15 (N72D+M38-K41Q,L45S,I67T,N79Y,E93A). Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:23-24.

N-810 A (TGRβRII-aglycosylated):

In one embodiment, the recombinant protein complex disclosed herein comprises N-810 A (TGRβRII-aglycosylated). In this embodiment, the polypeptide sequences of SEQ ID NO:25 and SEQ ID NO:26 are stabilized by hydrophobic or hydrophilic interactions to form the recombinant protein complex. SEQ ID NO:25 comprises, in a sequential manner, Leader Peptide, human αPDL1 scFv, IL15Rα-Fc, (G45)4 Linker, and human TGFβRII-N607Q,N631Q. SEQ ID NO: 26 comprises, in a sequential manner, Leader Peptide and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:25-26.

N-810 A (IL15-L455): In one embodiment, the recombinant protein complex disclosed herein comprises N-810 A (IL15-L455). In this embodiment, the polypeptide sequences of SEQ ID NO: 27 and SEQ ID NO: 28 are stabilized by hydrophobic or hydrophilic interactions to form the recombinant protein complex. SEQ ID NO:27 comprises, in a sequential manner, Leader Peptide, human αPDL1 scFv, IL15Rα-Fc, (G45)4 Linker, and human TGFβRII. SEQ ID NO:28 comprises, in a sequential manner, Leader Peptide and IL15 N72D-L45S. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:27-28.

N-810 B (TGFβRII dimer-aglycosylated/human αPD-L1/T×M): In one embodiment, the recombinant protein complex disclosed herein comprises TGFβRII dimer-aglycosylated/human αPD-L1/T×M. In this embodiment, the polypeptide sequences of SEQ ID NO:29 and SEQ ID NO:30 are stabilized by hydrophobic or hydrophilic interactions to form the recombinant protein complex. SEQ ID NO:29 comprises, in a sequential manner, Leader Peptide, human αPDL1 scFv, and IL15Rα-Fc. SEQ ID NO: 30 comprises, in a sequential manner, Leader Peptide, human TGFβRII dimer-N47Q,N71Q,N131Q,N198Q,N222Q,N282Q and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:29-30.

N-810 C (αPD-L1/TGFβRII dimer-aglycosylated/T×M): In one embodiment, the recombinant protein complex disclosed herein comprises N-810 C (αPD-L1/TGFβRII dimer-aglycosylated/T×M). In this embodiment, the polypeptide sequences of SEQ ID NO: 31 and SEQ ID NO: 32 are stabilized by hydrophobic or hydrophilic interactions to form the recombinant protein complex. SEQ ID NO: 31 comprises, in a sequential manner, Leader Peptide, human TGFβRII dimer-N47Q,N71Q,N131Q,N198Q,N222Q,N282Q, and IL15Rα-Fc. SEQ ID NO:32 comprises, in a sequential manner, Leader Peptide, human αPDL1 scFv, and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:31-32.

N-810 E (human αPD-L1/TGFβRII-aglycosylated/T×M): In one embodiment, the recombinant protein complex disclosed herein comprises N-810 E (human αPD-L1/TGFβRII-aglycosylated/T×M). In this embodiment, the polypeptide sequences of SEQ ID NO:33 and SEQ ID NO:34 are stabilized by hydrophobic or hydrophilic interactions to form the recombinant protein complex. SEQ ID NO:33 comprises, in a sequential manner, Leader Peptide, human TGFβRII-N47Q,N71Q,N131Q, and IL15Rα-Fc. SEQ ID NO:34 comprises, in a sequential manner, Leader Peptide, human αPDL1 scFv, and IL15 N72D. Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:33-34.

N-810D (IL15-N72D,L45S): In one embodiment, the recombinant protein complex disclosed herein comprises N-810D (IL15-N72D,L45S). In this embodiment, the polypeptide sequences of SEQ ID NO:35 and SEQ ID NO:36 are stabilized by hydrophobic or hydrophilic interactions to form the recombinant protein complex. SEQ ID NO:35 comprises, in a sequential manner, Leader Peptide, IL15Rα-Fc, (G45)4 Linker, and human TGFβRII. SEQ ID NO:36 comprises, in a sequential manner, Leader Peptide, human αPDL1 scFv/IL15 (N72D-L45S). Thus, the recombinant protein complex disclosed herein has preferably at least 80%, more preferably at least 85%, more preferably at least 90%, more preferably at least 95%, more preferably at least 99%, and most preferably 100% sequence identity to SEQ ID NOs:35-36.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Moreover, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Moreover, as used herein, the phrase “at least one of A and B” is intended to refer to ‘A’ and/or ‘B’, regardless of the nature of ‘A’ and ‘B’. For example, in some embodiments, ‘A’ may be single distinct species, while in other embodiments ‘A’ may represent a single species within a genus that is denoted ‘A’. Likewise, in some embodiments, ‘B’ may be single distinct species, while in other embodiments ‘B’ may represent a single species within a genus that is denoted ‘B’.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A recombinant protein complex comprising:
an interleukin-15 (IL-15) domain comprising an N72D mutation (IL-15N72D), a IL-15 receptor alpha sushi-binding domain (IL-15RαSu), an immunoglobulin Fc domain, and a mutated transforming growth factor-beta receptor type 2 (TGFβRII) domain, wherein the mutated TGFβRII domain comprises at least N->Q mutations in positions 47, 71, and 131;
the IL-15RαSu domain, the Fc domain, and the mutated TGFβRII domain are sequentially linked by amide bonds,
the IL-15 domain and/or the IL-15RαSu domain comprises a binding domain that specifically binds to a disease antigen, immune checkpoint molecule or immune signaling molecule, and
wherein the IL-15 domain binds to the IL-15RαSu domain to form the recombinant protein complex.

2. The recombinant protein complex of claim 1, wherein the immunoglobulin Fc domain is linked to a transforming growth factor-beta receptor type 2 (TGFβRII) domain via a linker molecule.

3. The recombinant protein complex of claim 1, wherein the binding domain comprises anti-programmed death ligand 1 (anti-PD-L1), and wherein the binding domain specifically binds to PD-L1.

4. The recombinant protein complex of claim 1, wherein the binding domain specifically binds to one or more molecules comprising: programmed death ligand 1 (PD-L1), programmed death 1 (PD-1), cytotoxic T-lymphocyte associated protein 4 (CTLA-4), cluster of differentiation 33 (CD33), cluster of differentiation 47 (CD47), glucocorticoid-induced tumor necrosis factor receptor (TNFR) family related gene (GITR), lymphocyte function-associated antigen 1 (LFA-1), tissue factor (TF), delta-like protein 4 (DLL4), single strand DNA or T-cell immunoglobulin and mucin-domain containing-3 (Tim-3).

5. The recombinant protein complex of claim 1, wherein the TGFβRII domain binds to transforming factor beta (TGFβ).

6. The recombinant protein complex of claim 1, wherein the mutated TGFβRII domain comprises SEQ ID NO: 2

7. A method of treating a tumor and/or an infectious disease in a subject in need thereof comprising administering to the subject an effective amount of a pharmaceutical composition comprising the recombinant protein complex of claim 1, thereby treating the tumor or infectious disease.

8. The method of claim 7, wherein the tumor comprises: glioblastoma, prostate cancer, hematological cancer, B-cell neoplasms, multiple myeloma, B-cell lymphoma, B cell non-Hodgkin lymphoma, Hodgkin's lymphoma, chronic lymphocytic leukemia, acute myeloid leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, prostate cancer, pancreatic cancer, colorectal cancer, ovarian cancer, non-small cell lung carcinoma, or squamous cell head and neck carcinoma.

9. The method of claim 7, optionally comprising administering to the subject one or more chemotherapeutic agents.

10. A method of inducing antibody-dependent cell-mediated cytotoxicity (ADCC) in a subject in need thereof, comprising administering to a subject in need thereof, an effective amount of a recombinant protein complex of claim 1.

11. An expression vector, comprising:
a first segment encoding an interleukin-15 (IL-15) domain comprising an N72D mutation (IL-15N72D);
a second segment encoding a polypeptide comprising a binding domain that specifically binds to a disease antigen, immune checkpoint molecule or immune signaling molecule, wherein the binding domain is linked to a IL-15 receptor alpha sushi-binding domain (IL-15RαSu) that is linked to an immunoglobulin Fc domain which is linked to a mutated transforming growth factor-beta receptor type 2 (TGFβRII) domain, wherein the mutated TGFβRII domain comprises at least N->Q mutations in positions 47, 71, and 131.

12. The expression vector of claim 11, wherein the vector is a viral vector, yeast vector, or bacterial vector.

13. The expression vector of claim 12, wherein the viral vector is a viral vector adenoviral vector.

14. The expression vector of claim 13, wherein the adenovirus has E1 and E2b genes deleted.

15. The expression vector of claim 11, wherein the immunoglobulin Fc domain is linked to a transforming growth factor-beta receptor type 2 (TGFβRII) domain via a linker molecule.

16. The expression vector of claim 11, wherein the binding domain comprises anti-programmed death ligand 1 (anti-PD-L1), and wherein the binding domain specifically binds to PD-L1.

17. The expression vector of claim 11, wherein the binding domain specifically binds to one or more molecules comprising: programmed death ligand 1 (PD-L1), programmed death 1 (PD-1), cytotoxic T-lymphocyte associated protein 4 (CTLA-4), cluster of differentiation 33 (CD33), cluster of differentiation 47 (CD47), glucocorticoid-induced tumor necrosis factor receptor (TNFR) family related gene (GITR), lymphocyte function-associated antigen 1 (LFA-1), tissue factor (TF), delta-like protein 4 (DLL4), single strand DNA or T-cell immunoglobulin and mucin-domain containing-3 (Tim-3).

18. The expression vector of claim 11, wherein the TGFβRII domain binds to transforming factor beta (TGFβ).

19. A method of treating a tumor and/or an infectious disease in a subject in need thereof comprising administering to the subject an effective amount of a pharmaceutical composition comprising the viral expression vector of claim 11.

20. The method of claim 19, wherein the tumor comprises: glioblastoma, prostate cancer, hematological cancer, B-cell neoplasms, multiple myeloma, B-cell lymphoma, B cell non-Hodgkin lymphoma, Hodgkin's lymphoma, chronic lymphocytic leukemia, acute myeloid leukemia, cutaneous T-cell lymphoma, T-cell lymphoma, a solid tumor, urothelial/bladder carcinoma, melanoma, lung cancer, renal cell carcinoma, breast cancer, gastric and esophageal cancer, prostate cancer, pancreatic cancer, colorectal cancer, ovarian cancer, non-small cell lung carcinoma, or squamous cell head and neck carcinoma.


SEQUENCE LISTING (36 sequences)

SEQ ID NO: 1 - protein, 136 aa, Artificial Sequence
  TGFbRII wild
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 136 aa, Artificial Sequence
  TGFbRII mutated
IPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPD

SEQ ID NO: 3 - protein, 715 aa, Artificial Sequence
  N-810 A -Leader Peptide/human aPDL1 scFv/IL15Ra-Fc/(G4S)4 Linker/human TGFbRII
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCPITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - protein, 133 aa, Artificial Sequence
  N-810 A -Leader Peptide/IL15(N72D)
MEWSWVFLFFLSVTTGVHSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 5 - protein, 558 aa, Artificial Sequence
  N-810 B Peptide/human aPDL1 scFv/IL15Ra-Fc
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 420 aa, Artificial Sequence
  Leader Peptide/human TGFbRII dimer/IL15(N72D)
MEWSWVFLFFLSVTTGVHSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 7 - protein, 603 aa, Artificial Sequence
  N-810 C (human aPDL1/TGFbRII dimer/TxM) - Leader Peptide/human TGFbRII dimer/IL15Ra-Fc
MEWSWVFLFFLSVTTGVHSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 375 aa, Artificial Sequence
  N-810 C (human aPDL1/TGFbRII dimer/TxM) Leader Peptide/human aPDL1 scFv/IL15(N72D)
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 9 - protein, 715 aa, Artificial Sequence
  N-810 (h2*?PDL1/TxM/TGFbRII-WT) Leader Peptide/human aPDL1 scFv/IL15Ra-Fc/(G4S)4 Linker/human TGFbRII
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - protein, 133 aa, Artificial Sequence; identical to SEQ ID NO: 4
  N-810 (h2*aPDL1/TxM/TGFbRII-WT) Leader Peptide/IL15(N72D)

SEQ ID NO: 11 - protein, 452 aa, Artificial Sequence
  N-810 E (human  PDL1/TGFbRII/TxM) Leader Peptide/human TGFbRII/IL15Ra-Fc
MEWSWVFLFFLSVTTGVHSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 375 aa, Artificial Sequence; identical to SEQ ID NO: 8
  N-810 E (human  PDL1/TGFbRII/TxM) Leader Peptide/human aPDL1 scFv/IL15(N72D)

SEQ ID NO: 13 - protein, 715 aa, Artificial Sequence
  N-810 A delta C Leader Peptide/human aPDL1 scFv/IL15Ra-Fc-C312S/(G4S)4
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 133 aa, Artificial Sequence; identical to SEQ ID NO: 4
  N-810 A delta C Leader Peptide/IL15(N72D)

SEQ ID NO: 15 - protein, 715 aa, Artificial Sequence
  N-810 A delta C (TGFbRII-aglycosylated) Leader Peptide/human aPDL1 scFv/IL15Ra-Fc-C312S /human TGFbRII-N607Q,N631Q,N691Q
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPD

SEQ ID NO: 16 - protein, 133 aa, Artificial Sequence; identical to SEQ ID NO: 4
  N-810 A delta C (TGFbRII-aglycosylated) Leader Peptide/IL15(N72D)

SEQ ID NO: 17 - protein, 473 aa, Artificial Sequence
  N-810 D Leader Peptide/IL15Ra-Fc/(G4S)4 Linker/human TGFbRII
MEWSWVFLFFLSVTTGVHSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 18 - protein, 375 aa, Artificial Sequence; identical to SEQ ID NO: 8
  N-810 D Leader Peptide/human aPDL1 scFv/IL15(N72D)

SEQ ID NO: 19 - protein, 715 aa, Artificial Sequence
  N-810 A (h2*aPDL1/TxM/TGRbRII-aglycosylated) Leader Peptide/human aPDL1 scFv/IL15Ra-Fc/(G4S)4 Linker/human TGF RII-N607Q,N631Q,N691Q
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPD

SEQ ID NO: 20 - protein, 133 aa, Artificial Sequence; identical to SEQ ID NO: 4
  N-810 A (h2*aPDL1/TxM/TGRbRII-aglycosylated) Leader Peptide/IL15(N72D)

SEQ ID NO: 21 - protein, 710 aa, Artificial Sequence
  N-810 A delta Hinge Leader Peptide/human aPDL1 scFv/IL15Ra-Fc/(G4S)4 Linker/human TGFbRII
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 22 - protein, 133 aa, Artificial Sequence; identical to SEQ ID NO: 4
  N-810 A delta Hinge Leader Peptide/IL15(N72D)

SEQ ID NO: 23 - protein, 715 aa, Artificial Sequence; identical to SEQ ID NO: 9
  N-810 A (IL15-M38) Leader Peptide/human aPDL1 scFv/IL15Ra-Fc/(GS4)4 Linker/human TGFbRII

SEQ ID NO: 24 - protein, 133 aa, Artificial Sequence
  N-810 A (IL15-M38) Leader Peptide/IL15(N72D+M38-K41Q,L45S,I67T,N79Y,E93A)
MEWSWVFLFFLSVTTGVHSNWVNVISDLKKIEDLIQSMHIDATLYTESDVHPSCKVTAMQCFLSELQVISLESGDASIHDTVENLTILANDSLSSNGYVTESGCKECEELEAKNIKEFLQSFVHIVQMFINTS

SEQ ID NO: 25 - protein, 715 aa, Artificial Sequence
  N-810 A (TGRbRII-aglycosylated) Leader Peptide/human aPDL1 scFv/IL15Ra-Fc/(G4S)4 Linker/human TGF RII-N607Q,N631Q
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYNTSNPD

SEQ ID NO: 26 - protein, 133 aa, Artificial Sequence; identical to SEQ ID NO: 4
  N-810 A (TGRbRII-aglycosylated) Leader Peptide/IL15(N72D)

SEQ ID NO: 27 - protein, 715 aa, Artificial Sequence; identical to SEQ ID NO: 9
  N-810 A (IL15-L45S) Leader Peptide/human aPDL1 scFv/IL15Ra-Fc/(G4S)4 Linker/human TGFbRII

SEQ ID NO: 28 - protein, 133 aa, Artificial Sequence
  N-810 A (IL15-L45S) Leader Peptide/IL15(N72D-L45S)
MEWSWVFLFFLSVTTGVHSNWVNVISDLKKIEDLIQSMHIDATLYTESDVHPSCKVTAMKCFLSELQVISLESGDASIHDTVENLIILANDSLSSNGNVTESGCKECEELEEKNIKEFLQSFVHIVQMFINTS

SEQ ID NO: 29 - protein, 558 aa, Artificial Sequence; identical to SEQ ID NO: 5
  N-810 B (TGFbRII dimer-aglycosylated/human aPD-L1/TxM) Leader Peptide/human aPDL1 scFv/IL15Ra-Fc

SEQ ID NO: 30 - protein, 420 aa, Artificial Sequence
  N-810 B (TGFbRII dimer-aglycosylated/human aPD-L1/TxM) Leader Peptide/human TGF RII dimer-N47Q,N71Q,N131Q,N198Q,N222Q,N282Q/IL15(N72D)
MEWSWVFLFFLSVTTGVHSIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPDGGGGSGGGGSGGGGSIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPDNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 31 - protein, 603 aa, Artificial Sequence
  N-810 C (aPD-L1/TGFbRII dimer-aglycosylated/ TxM) Leader Peptide/human TGF RII dimer-N47Q,N71Q,N131Q,N198Q,N222Q,N282Q/IL15Ra-Fc
MEWSWVFLFFLSVTTGVHSIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPDGGGGSGGGGSGGGGSIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPDITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 375 aa, Artificial Sequence; identical to SEQ ID NO: 8
  N-810 C (aPD-L1/TGFbRII dimer-aglycosylated/ TxM) Leader Peptide/human aPDL1 scFv/IL15(N72D)

SEQ ID NO: 33 - protein, 452 aa, Artificial Sequence
  N-810 E (human aPDL1/TGRbRII-aglycosylated/ TxM) Leader Peptide/human TGF RII-N47Q,N71Q,N131Q/IL15Ra-Fc
MEWSWVFLFFLSVTTGVHSIPPHVQKSVNNDMIVTDNNGAVKFPQLCKFCDVRFSTCDNQKSCMSQCSITSICEKPQEVCVAVWRKNDEQITLETVCHDPKLPYHDFILEDAASPKCIMKEKKKPGETFFMCSCSSDECNDNIIFSEEYQTSNPDITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 375 aa, Artificial Sequence; identical to SEQ ID NO: 8
  N-810 E (human aPDL1/TGRbRII-aglycosylated/ TxM) Leader Peptide/human aPDL1 scFv/IL15(N72D)

SEQ ID NO: 35 - protein, 473 aa, Artificial Sequence; identical to SEQ ID NO: 17
  N-810D (IL15-N72D,L45S) Leader Peptide/IL15Ra- Fc/(G4S)4 Linker/human TGFbRII

SEQ ID NO: 36 - protein, 489 aa, Artificial Sequence
  N-810D (IL15-N72D,L45S) Leader Peptide/human aPDL1 scFv/IL15(N72D-L45S)
MEWSWVFLFFLSVTTGVHSNIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKRGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSNWVNVISDLKKIEDLIQSMHIDATLYTESDVHPSCKVTAMKCFLSELQVISLESGDASIHDTVENLIILANDSLSSNGNVTESGCKECEELEEKNIKEFLQSFVHIVQMFINTSNWVNVISDLKKIEDLIQSMHIDATLYTESDVHPSCKVTAMKCFLSELQVISLESGDASIHDTVENLIILANDSLSSNGNVTESGCKECEELEEKNIKEFLQSFVHIVQMFINTS